(19) United States
(12) Patent Application Publication
(10) Pub. No.: US 2020/0263220 A1
(43) Pub. Date: Aug. 20, 2020

(54) METHOD FOR INCREASING THE HETEROGENEITY OF O-GLYCOSYLATION OF RECOMBINANT FACTOR VII

(71) Applicant: CSL BEHRING RECOMBINANT FACILITY AG, Bem (CH)

(72) Inventors: Yih Yean LEE, Altona, Victoria (AU); Vanessa SANDFORD, Trentham, Victoria (AU)

(21) Appl. No.: 16/061,457

(22) PCT Filed: Dec. 14, 2016
(86) PCT No.: PCT/EP2016/080899

§ 371 Date: Jun. 12, 2018

(30) Foreign Application Priority Data
Dec. 15, 2015 (EP) 15200175.6

(51) Int. Cl. C12P 21/00; C12N 5/00; C12N 5/071; C07K 14/745

(52) U.S. Cl. CPC C12P 21/005; C12N 5/005; A61K 38/00; C07K 14/745; C12N 2500/34; C12N 5/0682

(57) ABSTRACT

The present invention relates to a method for increasing the O-glycan heterogeneity of recombinant Factor VII (FVII). In particular, the present invention relates to a method for reducing the occurrence of Xyl-Xyl-Glc-glycosylation of FVII. The present invention further relates to uses, compositions of matter, recombinant FVII prepared by the method of the invention, pharmaceutical compositions comprising recombinant FVII prepared by the method of the invention and medical uses of recombinant FVII prepared by the method of the invention.

### FIELD OF THE INVENTION

The present invention relates to a method for increasing the 0-glycan heterogeneity of recombinant Factor VII (FVII). In particular, the present invention relates to a method for reducing the occurrence of Xyl-Xyl-Glc-glycosylation of FVII. The present invention further relates to uses, compositions of matter, recombinant FVII prepared by the method of the invention, pharmaceutical compositions comprising recombinant FVII prepared by the method of the invention and medical uses of recombinant FVII prepared by the method of the invention.

### BACKGROUND OF THE INVENTION

FVII is a single-chain glycoprotein with a molecular weight of about 50 kDa, which is secreted by liver cells into the blood stream as an inactive zymogen of 406 amino acids. FVII is O-glycosylated at Ser52 and Ser60. At position Ser52 three different glycan structures, glucose- (Glc-), xylose-glucose- (Xyl-Glc-) and xylose-xylose-glucose- (Xyl-Xyl-Glc-) were identified. In plasma derived FVII, the three different glycan structures at Ser52 are present in approximately equal amounts. In contrast, recombinantly expressed FVII is predominantly modified with Xyl-Xyl-Glc- at Ser52. Accordingly, the glycosylation pattern of plasma derived FVII is more heterogenic than the glycosylation pattern of recombinant FVII (Bjoern et al., 1991, The Journal of Biological Chemistry, Vol. 266, No. 17, pp. 11051-11057 and Fenaille et al., 2008, Glycoconjugate Journal, 25:827-842). Further posttranslational modifications of FVII include hydroxylation at Asp63 and N-glycosylation at Asn145 and Asn322. FVII contains 10 γ-carboxy-glutamic acid residues (positions 6, 7, 14, 16, 19, 20, 25, 26, 29, and 35) localized in the N-terminal Gla-domain of the protein. However, FVII with 9 Gla residues is the predominant species. The Gla residues require vitamin K for their biosynthesis. Located C-terminal to the Gla domain are two epidermal growth factor domains followed by a trypsin-type serine protease domain.

FVII is used mainly in its activated form for the treatment of various bleeding disorders. For therapeutic uses, recombinant FVII is advantageous over plasma-derived FVII, since plasma-derived FVII has a potentially higher risk of pathogen contamination and is associated with high efforts and expense as its process of preparation is dependent on human plasma donors. Recombinant FVII can be derived by expressing FVII in mammalian cell culture and subsequent purification of FVII. Recombinant FVII products on the market include NovoSeven® (Novo Nordisk) and AryoSeven™ (Aryogen).

The glycosylation pattern of a protein can alter various features of a protein, such as activity, solubility immunogenicity, bioavailability and half-life. The glycosylation pattern of FVII has been shown to be crucial for its optimal function (Morfini et al., 2012, Haemophilia, 18, 431-436 and Bjoern et al., 1991, The Journal of Biological Chemistry, Vol. 266, No. 17, pp. 11051-11057). Hence, a recombinant FVII preparation with a post-translational modification pattern similar or identical to plasma derived FVII is desirable for therapeutic application.

Methods for altering the glycosylation pattern of recombinant proteins have been disclosed in the prior art (e.g. Kildegaard et al., 2015, Biotechnology and Bioengineering, 2015 Jul. 29. doi: 10.1002/bit.25715; Wong et al., 2010, Biotechnology and Bioengineering, Vol 107, No 2, pp 321-336 and Liu et al. 2015, World J Microbiol Biotechnol, 31:1147-1156). WO 2014/159259 discloses a method for modulating high mannose glycan species on a recombinant protein during a mammalian cell culture process by limiting the amount of glucose in the cell culture medium and supplementing the cell culture medium with galactose or sucrose. WO 2015/026846 pertains to a cell culture medium comprising media supplements that are shown to control recombinant protein glycosylation and/or cell culture in a controlled or modulated (shifted) temperature to control recombinant protein glycosylation and/or cell culture with controlled or modulated seed density to control recombinant protein glycosylation. It further pertains to a method of controlling or manipulating glycosylation of a recombinant protein of interest in a large scale cell culture. WO 2012/149197 discloses methods for modulating the glycosylation profile of recombinantly expressed proteins. In particular, it discloses methods of controlling the galactosylation profile of recombinantly expressed proteins by supplementing production medium, e.g., a hydrolysate-based or a chemically defined medium, with manganese and/or D-galactose. Further methods for altering a protein's glycosylation pattern are disclosed in WO 2013/114164, in WO 2004/058944, and in WO 2004/008100.

WO 2005/111225 discloses methods for enzymatically altering the glycosylation pattern of FVII. Further, FVII preparations with increased levels of Xyl-Xyl-Glc- glycosylation at position Ser52 of FVII are provided. Accordingly, the disclosed methods aim to reduce O-glycan heterogeneity of FVII and the resulting FVII is different from the natural form as found in human plasma.

In contrast, the present invention relates to a method for increasing O-glycan heterogeneity of FVII at Ser52. Thereby, the present invention provides a method that allows preparing FVII with a glycosylation pattern that more closely resembles the glycosylation pattern of plasma derived FVII. Recombinant replacements of human protein therapeutics should resemble as closely as possible their natural counterpart to reduce the risk of adverse events as much as possible. For therapeutic application, it is highly desirable to provide FVII with post-translational modifications similar to plasma derived FVII. The method according to the present invention fulfills this unmet need in the art.

### SUMMARY OF THE INVENTION

The present invention relates to a method for increasing the O-glycan heterogeneity of FVII. Further, the provided method is for reducing the occurrence of Xyl-Xyl-Glc-glycosylation of FVII.

Recombinant FVII predominantly exhibits Xyl-Xyl-Glc-glycosylation at Ser52. By applying the provided method, recombinant FVII can be brought closer to the natural state, where the three forms Xyl-Xyl-Glc-, Xyl-Glc- and Glc- occur in about equal amounts on Ser52.

It has been surprisingly found that the presence of galactose in the cell culture medium during expression of FVII in host cells results in increased heterogeneity of O-glycan structures at position Ser52 of recombinant FVII. In particular, the method of the present invention results in a decrease of Xyl-Xyl-Glc-glycosylation at position Ser52 of recombinant FVII.

In one embodiment, the present invention provides a method for increasing heterogeneity of O-glycan structures at Ser52 of recombinant FVII comprising the following steps:

a) providing host cells comprising an expression system expressing recombinant FVII,
b) culturing the cells in a cell culture medium comprising galactose, and
c) separating and/or isolating and/or purifying the recombinant FVII from the cell culture,
wherein Ser52 of recombinant FVII is the residue corresponding to Ser52 of SEQ ID NO:1.

In a further embodiment, the present invention provides a method for reducing the occurrence of Xyl-Xyl-Glc-glycosylation at Ser52 of recombinant FVII comprising the following steps:

a) providing host cells comprising an expression system expressing recombinant FVII,
b) culturing the cells in a cell culture medium comprising galactose, and
c) separating and/or isolating and/or purifying the recombinant FVII from the cell culture,
wherein Ser52 of recombinant FVII is the residue corresponding to Ser52 of SEQ ID NO:1.

In another embodiment, the present invention provides a method for increasing O-glycan heterogeneity at Ser52 of recombinant FVII and for reducing the occurrence of Xyl-Xyl-Glc-glycosylation at Ser52 of recombinant FVII comprising the following steps:

a) providing host cells comprising an expression system expressing recombinant FVII,
b) culturing the cells in a cell culture medium comprising galactose, and
c) separating and/or isolating and/or purifying the recombinant FVII from the cell culture,
wherein Ser52 of recombinant FVII is the residue corresponding to Ser52 of SEQ ID NO:1.

In one embodiment, galactose is provided in the cell culture at a concentration effective to increase heterogeneity of O-glycan structures at position Ser52 of FVII. In another embodiment, galactose is provided in the cell culture at a concentration effective to reduce the occurrence of Xyl-Xyl-Glc-glycosylation at position Ser52 of FVII.

In one embodiment, galactose is provided in the cell culture at a concentration of at least 1 mM, preferably at least 2.5 mM. In a further preferred embodiment, galactose is provided in the cell culture at a concentration of at least 10 mM. In another embodiment, galactose is provided in the cell culture at a concentration between 1 mM and 60 mM. In a preferred embodiment, galactose is provided in the cell culture at a concentration between 2.5 mM and 60 mM. In a further preferred embodiment, galactose is provided in the cell culture at a concentration between 10 mM and 60 mM, more preferably between 10 mM and 40 mM.

Preferably, the galactose concentration in the cell culture is about 20 mM. In one embodiment, galactose is maintained above a concentration of 10 mM in the cell culture. The cell culture medium may be protein-free or chemically defined.

In a further embodiment, the cell culture is a fed-batch culture. Galactose may be present in the basal cell culture medium. Alternatively or additionally, galactose may be present in the feed medium. In one embodiment, the galactose concentration in the basal medium is at least 1 mM, preferably at least 2.5 mM, more preferably at least 10 mM. In a further embodiment, the galactose concentration in the basal medium is between 2.5 mM and 60 mM, preferably between 10 and 60 mM, more preferably between 10 and 40 mM. A feed medium comprising galactose is added in an amount sufficient to maintain the galactose concentration the cell culture above a threshold level of 1 mM, preferably above a threshold level of 2.5 mM, more preferably above a threshold level of 10 mM. The feed medium may be added continuously or periodically during cell culture. Typically, the total feed added over the culture period is between 10% and 30% of the entire culture volume, for example about 20% of the entire culture volume.

In another embodiment, the cell culture is a perfusion culture. Galactose may be present in the basal cell culture medium. Alternatively or additionally, galactose may be present in the perfusion medium. In one embodiment, the galactose concentration in the basal medium is at least 1 mM, preferably at least 2.5 mM, more preferably at least 10 mM. In a further embodiment, the galactose concentration in the basal medium is between 2.5 mM and 60 mM, preferably between 10 and 60 mM, more preferably between 10 and 40 mM. A perfusion medium comprising galactose is added in an amount sufficient to maintain the galactose concentration in the cell culture above a threshold level of 1 mM, preferably above a threshold level of 2.5 mM, more preferably above a threshold level of 10 mM. The perfusion medium may be added continuously or semi-continuously during cell culture. Typical dilution rates for perfusion cultures can be anywhere from 0.5 to 2 culture volumes per day.

In a preferred embodiment, the host cells expressing FVII are CHO cells. Further, the recombinant FVII may be a fusion protein. In a preferred embodiment, the FVII is an albumin fusion protein.

In one embodiment, the occurrence of Xyl-Xyl-Glc-glycosylation at Ser52 of recombinant FVII prepared according to the method of the invention is below 80%, preferably below 70%.

Another embodiment is the use of a cell culture medium comprising galactose for increasing O-glycan heterogeneity at Ser52 of recombinant FVII and/or for reducing the occurrence of Xyl-Xyl-Glc-glycosylation at Ser52 of recombinant FVII, wherein Ser52 of recombinant FVII is the residue corresponding to Ser52 of SEQ ID NO:1.

Also provided is a recombinant FVII composition produced in CHO cells, wherein the occurrence of Xyl-Xyl-Glc-glycosylation at Ser52 of recombinant FVII is below 80%, preferably below 70%, wherein Ser52 of recombinant FVII is the residue corresponding to Ser52 of SEQ ID NO:1.

A pharmaceutical composition comprising the recombinant FVII prepared according to the provided method and a pharmaceutically acceptable excipient is also provided herein. Further provided are a composition of matter, a bioreactor comprising a composition of matter and medical uses.

### DESCRIPTION OF THE FIGURES

FIG. 1: FIG. 1A: TIC (total ion chromatogram) and BPC (base peak chromatogram) of the and corresponding deconvoluted and annotated spectrum highlighting the major glycoforms.

FIG. 2: Xyl-Xyl-Glc at Ser52 of FVII expressed in cell culture medium comprising different concentrations of galactose (0 mM, 1 mM, 2.5 mM, 5 mM, 10 mM, 20 mM, 30 mM and 40 mM).

FIG. 3: Overview of experimental outline of experiment shown in Example 2.

FIG. 4: Xyl-Xyl-Glc at Ser52 of FVII expressed in cell culture medium in absence or presence of galactose (20 mM) in large scale bioreactor experiments.

FIG. 5: SEQ ID NO:1 (amino acid sequence of FVIIa).

### DETAILED DESCRIPTION OF THE INVENTION

The term “about” as used herein refers to a range of values similar to the stated reference value. In particular, the term about refers to a range of values that falls within 10%, 5%, 3% above and 10%, 5%, 3% below the reference value.

The term “Factor VII” or “FVII” as used herein encompasses wild-type Factor VII and its activated form Factor VIIa, and variants of Factor VII and Factor VIIa that exhibit substantially the same or improved biological activity relative to wild-type Factor VII or Factor VIIa. The term “Factor VII” is thus intended to encompass Factor VII polypeptides in their uncleaved (zymogen) form, as well as those that have been proteolytically processed to yield their respective bioactive forms, which may be designated Factor VIIa. Typically, FVII is converted to its active form Factor VIIa (FVIIa) by proteolysis of the single peptide bond at Arg152-Ile153 leading to the formation of two polypeptide chains, a N-terminal light chain (24 kDa) and a C-terminal heavy chain (28 kDa), which are held together by one disulfide bridge. In contrast to other vitamin K-dependent coagulation factors, no activation peptide, which is cleaved off during activation of these other vitamin K-dependent coagulation factors, has been described for FVII. The Arg152-Ile153 cleavage site and some amino acids downstream show homology to the activation cleavage site of other vitamin K-dependent polypeptides. Essential for attaining the active conformation of Factor VIIa is the formation of a salt bridge after activation cleavage between Ile153 and Asp343. Activation cleavage of Factor VII can be achieved in vitro by Factor Xa, Factor XIIa, Factor IXa, Factor VIIa, Factor Seven Activating Protease (FSAP) and thrombin. Mollerup et al., 1995 (Biotechnol. Bioeng. 48:501-505) reported that some cleavage also occurs in the heavy chain at Arg290 and or Arg315.

Also encompassed is recombinant Factor VII, or variants thereof, for example, in which one or more amino acid deletions, additions, and/or substitutions have been introduced to modulate (e.g., increase, decrease) at least one biological activity of the protein. Unless otherwise specified, the FVII referred to herein may be unmodified or may exhibit post-translational modifications. Further encompassed are FVII fusion proteins, such as a FVII-albumin fusion. FVII may be human FVII. Also included are Factor VII proteins or FVII-related proteins from other organisms, such as other mammals.

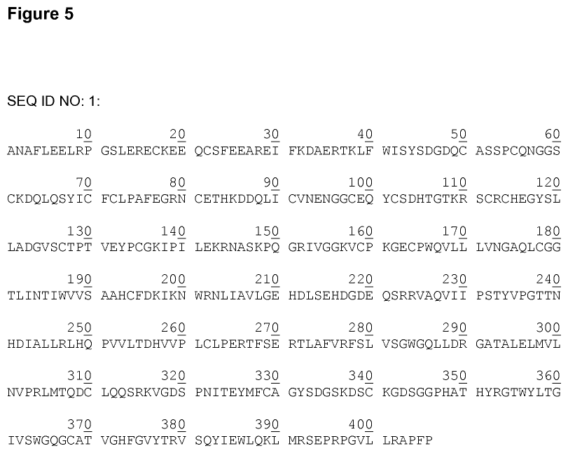

The numbering of the amino acid residues of recombinant FVII as used herein refers to the amino acid numbering of human wild-type FVII (SEQ ID NO:1, FIG. 5). Accordingly, the amino acid numbering indicated herein generally refers to the amino acid residues corresponding to the indicated residue of SEQ ID NO:1.

“Ser52” or “Serine 52” as used herein refers to the amino acid residue at position 52 counted from the N-terminus of SEQ ID NO:1. If the FVII is a FVII variant or a fusion protein, Ser52 may not be at position 52 of said variant or fusion protein, but it is the residue corresponding to Ser52 of SEQ ID NO:1 (FIG. 5).

Ser60” or “Serine 60” as used herein refers to the amino acid residue at position 60 counted from the N-terminus of SEQ ID NO:1. If the FVII is a FVII variant or a fusion protein, Ser60 may not be at position 60 of said variant or fusion protein, but it is the residue corresponding to Ser60 of SEQ ID NO:1 (FIG. 5).

FVII plays an important role in promoting blood coagulation. The current model of coagulation states that the physiological trigger of coagulation is the formation of a complex between tissue Factor (TF) and FVII on the surface of TF expressing cells, which are normally located outside the vasculature. This leads to the activation of Factor IX and Factor X ultimately generating some thrombin. In a positive feedback loop thrombin activates Factor VIII and Factor IX, the so-called “intrinsic” arm of the blood coagulation cascade, thus amplifying the generation of Factor Xa, which is necessary for the generation of the full thrombin burst to achieve complete hemostasis. It was shown that by administering supraphysiological concentrations of Factor VIIa hemostasis is achieved bypassing the need for Factor Villa and Factor IXa. The cloning of the cDNA for Factor VII (U.S. Pat. No. 4,784,950) made it possible to develop activated Factor VII as a pharmaceutical. Factor VIIa was successfully administered for the first time in 1988.

FVII is used in the treatment of Haemophilia A and B in patients who developed inhibitors against replacement factors. Haemophilia A and B are inherited coagulation disorders. They result from a chromosome X-linked deficiency of blood coagulation Factor VIII (Haemophilia A) or from a chromosome X-linked deficiency of blood coagulation Factor IX (Haemophilia B) and affect almost exclusively males with an incidence between one and two individuals per 10,000. The X-chromosome defect is transmitted by female carriers who are not themselves clinically symptomatic. The clinical manifestation of haemophilia A and B is an increased bleeding tendency. The goal of therapy for haemophilia is to treat or prevent hemorrhage, thereby reducing disabling joint and tissue damage, and improving quality of life. In both, haemophilia A and in haemophilia B, the most serious medical problem in treating the disease is the generation of inhibitory alloantibodies against the replacement factors. Up to 30% of all haemophilia A patients develop inhibitory antibodies to Factor VIII. Inhibitory antibodies to Factor IX occur to a lesser extent but with more severe consequences, as they are less susceptible to immune tolerance induction therapy and have a higher potential to trigger allergic reactions when binding to Factor IX. The treatment for patients with haemophilia A (FVIII deficiency) or haemophilia B (Factor IX deficiency) who have developed inhibitory antibodies (Congenital Haemophilia with Inhibitors, CHwI) to FVIII or Factor IX (especially high titer inhibitors) is challenging, since normal replacement with Factor VIII or IX is not effective.

FVII can also be used as therapy to treat bleeding associated with perioperative and traumatic blood loss in subjects with normal coagulation systems. For example, FVII can be administered to a patient to promote coagulation and reduce blood loss associated with trauma and surgery and, further, reduce the requirement for blood transfusion. FVII can further be used in the treatment of acquired haemophilia, congenital FVII deficiency and Glanzmann's thrombasthenia.

The term “increasing O-glycan heterogeneity at Ser52 of recombinant FVII” as used herein relates to an increase of the variability of the occurrence of glycan structures on Ser52 of FVII. Three different glycan structures (Glc-, Xyl-Glc- and Xyl-Xyl-Glc-) occur on Ser52 of FVII. By increasing the O-glycan heterogeneity of FVII, the occurrence of the different glycan structures on Ser52 is shifted from one (or two) predominately occurring structures to a more equal distribution of the three different structures.

The term “reducing the occurrence of Xyl-Xyl-Glc-glycosylation at Ser52 at recombinant FVII” as used herein relates to a decrease of the occurrence of Xyl-Xyl-Glc-glycosylation on Ser52 of FVII. By reducing the occurrence of Xyl-Xyl-Glc-glycosylation at Ser52, the occurrence of the different glycan structures on Ser52 is shifted from one predominately occurring structure (Xyl-Xyl-Glc-) to a more equal distribution of the three different structures (Glc-, Xyl-Glc- and Xyl-Xyl-Glc-). For example, the occurrence of Xyl-Xyl-Glc- at Ser52 of recombinant FVII may be reduced below 80%, below 70% or even below 60%.

The term “galactose” as used herein relates to open-chain and cyclic galactose isomers.

The term “albumin” as used herein, includes polypeptides of the albumin family of proteins such as human serum albumin, including variants and derivatives thereof, such as genetically engineered or chemically modified albumin variants and fragments of albumin proteins. Human serum albumin (HSA, or HA), is a protein of 585 amino acids in its mature form, and is responsible for a significant proportion of the osmotic pressure of serum and also functions as a carrier of endogenous and exogenous ligands. Among other benefits, fusion to HSA or a fragment or variant thereof can increase the shelf-life, plasma half-life, and/or therapeutic activity of its fusion partner. Albumin may be fused to its fusion partner to the N-terminus and/or to the C-terminus. If the fusion protein is FVII the albumin is preferably fused to the C-terminus of FVII. The albumin portion of a fusion protein may also be derived from any vertebrate, especially any mammal. The albumin portion of the albumin-fusion protein may be from a different animal than the other protein portion of the fusion protein.

The term “glycan” as used herein refers to oligosaccharide structures that are linked to serine/threonine residues (O-glycosylation) or asparagine residues (N-glycosylation). The glycan may comprise one or more saccharide moieties. O-glycosylation typically occurs at the motif Cys-X1-Ser/Thr-X2-Pro-Cys. Such O-glycosylation motif leads to the O-glycosylation of Ser52 of FVII.

The term “host cells” as used herein refers to cells from any cell line suitable for protein expression. The cell line may be of mammalian origin. Non-limiting examples of suitable mammalian cell lines are HEK 293 cells (human embryonic kidney 293 cells), BHK cells (baby hamster kidney cells), COS-1 cells, 3T3 cells, CHO cells (Chinese hamster ovary cells), hybridoma cells, NS0 mouse myeloma cells, NS1 mouse myeloma cells, Sp2/0 mouse myeloma cells, PER.C6 human retinoblastoma cells, Vero African Green Monkey Kidney cells and MDCK Mardin-Darby Canine Kidney cells.

The term “cell culture medium” as used herein is a medium to culture mammalian cells comprising a minimum of essential nutrients and components required for cell growth such as vitamins, trace elements, salts, amino acids, carbohydrates in a preferably buffered medium. Non-limiting examples for such a cell culture medium are commercially available media and proprietary media. The cell culture medium can be a basal cell culture medium. The cell culture medium can also be a basal cell culture medium to which the feed medium or other additives have been added.

The term “basal medium” as used herein is a cell culture medium to culture mammalian cells. It refers to the medium in which the cells are cultured from the start of a cell culture. It is not used as an additive to another medium, however various components may be added to the medium. To the basal medium optionally further additives, feed medium or perfusion medium may be added during cell culture. The basal cell culture medium provides generally nutrients required for cell growth such as carbon sources, amino acids, vitamins and glucose.

The term “feed medium” as used herein relates to a concentrated nutrient formulation used as a feed in a cell culture. Feed medium is added to a cell culture during culturing cells. It is provided as a concentrated feed medium to avoid dilution of the cell culture medium. The feeding rate is to be understood as an average feeding rate over the feeding period. A feed medium usually has higher concentrations of the components that are to be replenished than the basal medium. The feed medium replenishes components that are consumed during cell culture, such as amino acids and carbohydrates. The feed may be added in different modes such as continuously or periodically. The feed medium may be added daily, but may also be added more frequently, such as twice daily or less frequently, such as every second day.

The term “perfusion medium” as used herein relates to a medium suitable to replace cell culture medium that has been removed from a cell culture during culturing cells. Perfusion medium may have a similar or identical formulation as the basal medium. However, in the perfusion medium, several components may be present in higher or lower concentration or even be absent in comparison to the basal medium. The perfusion medium may also comprise components that are not present in the basal medium.

The cell culture medium, the basal medium, the feed medium and/or the perfusion medium may be serum-free, chemically defined, free of any proteins from human or animal origin and/or protein-free.

A “serum-free medium” as used herein refers to a cell culture medium for in vitro cell culture, which does not contain serum from animal origin. This is preferred as serum may contain contaminants or pathogens from said animal. Further, serum lacks a clear definition and may vary in its composition from batch to batch.

A “chemically defined medium” as used herein refers to a cell culture medium for in vitro cell culture, in which all components and preferably their respective amounts are known. More specifically, it does not contain any undefined supplements such as animal serum or plant hydrolysates. It may however comprise hydrolysates if all components have been analyzed and the exact composition of the hydrolysate is known.

A medium “free of proteins from animal or human origin” as used herein refers to a cell culture medium that does not contain any protein components from an animal or human source. However, such medium may comprise recombinant proteins derived from, e.g., expression cell culture or bacterial expression, like recombinant insulin.

A “protein-free medium” as used herein refers to a cell culture medium for in vitro cell culture comprising no proteins, except for proteins produced by the cell to be cultured, wherein protein refers to polypeptides of any length, but excludes single amino acids, dipeptides or tripeptides.

The term “cell culture” or “cultivation” includes cell cultivation and fermentation processes in all scales (e.g. from micro titer plates to large-scale bioreactors, i.e. from sub mL-scale to >1000 L scale), in all different process modes (e.g. batch, fed-batch, perfusion), in all process control modes (e.g. non-controlled, fully automated and controlled systems with control of e.g. pH, temperature, oxygen content), in all kind of fermentation systems (e.g. single-use systems, stainless steel systems, glass ware systems). Cell culture occurs at conditions (temperature, oxygen supply etc.) that are established for the respective cell lines used. The cells may be agitated or shaken to increase oxygenation and/or dispersion of nutrients during cultivation.

The term “fed-batch” as used herein relates to a cell culture in which the cells are fed continuously or periodically with a feed medium containing nutrients. The feeding may start shortly after starting the cell culture on day 0 or more typically one, two or three days after starting the culture. Feeding may follow a given schedule, such as every day, every two days etc. Alternatively, the culture may be monitored for cell growth, nutrients or toxic by-products and feeding may be adjusted accordingly. In general, the following parameters are often determined on a daily basis and cover the viable cell concentration, product concentration and metabolites such as glucose, galactose, pH, osmolarity (a measure for salt content) and ammonium (growth inhibitor that negatively affects the growth rate). Compared to batch cultures (where no feeding occurs), higher product titers can be achieved in the fed-batch mode. Typically, a fed-batch culture is stopped at some point and the cells and/or the protein of interest in the medium are harvested.

The term “perfusion culture” as used herein refers to a cell culture in which perfusion medium is added continuously or semi-continuously during cell culture. The addition may start shortly after starting the cell culture on day 0 or one or more days after starting the culture. A portion of the cells, the cell culture medium and/or components in the medium are harvested continuously or semi-continuously during cell culture. Harvesting may start when the perfusion medium addition starts. The harvested components (e.g. proteins) may optionally be purified. The amount of perfusion medium added to a cell culture usually depends on the amount of cell culture medium removed from the culture during harvesting. The culture may be monitored for cell growth, nutrients or toxic by-products and the perfusion rate (amount of perfusion medium added over time) may be adjusted accordingly.

Methods for introducing DNA encoding FVII into host cell in order to achieve FVII expression are known from the prior art (e.g., Kim et al., Anal Bioanal Chem (2010), 379:3173-3178). The DNA encoding FVII may further encode regulatory elements. Suitable elements should be selected based on the host cell. Preferred methods of transfection include the Lipofectamine® method, calcium phosphate precipitation and electroporation. The present invention can be carried out with transiently transfected or stably transfected host cells. In a preferred embodiment, the host cells are stably transfected with DNA encoding FVII.

In one embodiment the FVII is human FVII. In a further embodiment, the FVII is a fusion protein. In a further preferred embodiment, the FVII is an albumin fusion protein. A FVII albumin fusion protein is described, e.g., in WO 2007/090584.

In one embodiment, the cell culture is a batch culture. The culture is inoculated with an appropriate number of host cells and basal medium. In a batch culture, the cells grow in the basal medium throughout cultivation.

In another embodiment, the cell culture is a fed-batch culture. The culture is inoculated with an appropriate number of host cells and basal medium. Feed medium is added during cultivation in order to replenish nutrients and/or supplements. In one embodiment, the feed is added continuously. In another embodiment, the feed is added periodically. The feed may be added daily between days 3 and 9 of the cell culture. Further, a total amount of 20% of the starting volume may be added to the cell culture. In one embodiment, the feed is started on day 3 from the time point of inoculating the culture. Further, multiple different feeds comprising different nutrients or supplements may be added independently of each other to the cell culture. In one embodiment, the nutrient or supplement status of the culture is monitored throughout cultivation and the feed is added depending on the requirements of the culture.

In another embodiment, the cell culture is a perfusion culture. The culture is inoculated with an appropriate number of host cells and basal medium. Perfusion medium is added during cultivation in order to replenish nutrients, to remove toxic side products and/or supplements and/or in order to compensate for medium removed by harvesting. In one embodiment, the perfusion medium is added continuously. The perfusion medium may be added at a rate of 0.5 to 2 culture volumes per day. In another embodiment, the perfusion medium is added semi-continuously.

In a further embodiment, the galactose concentration of the culture is monitored and the galactose containing feed or galactose containing perfusion medium is added in order to maintain the galactose concentration in the culture medium above a threshold level.

In a preferred embodiment, the galactose concentration in the cell culture is maintained above the threshold level of 1 mM, preferably above the threshold level of 2.5 mM, most preferably above a threshold level of 10 mM.

In another preferred embodiment, the galactose concentration is maintained above the threshold level for a time period of 7-14 days, preferably of 8-12 days, more preferably of 9-11 days. In one embodiment, the galactose concentration is maintained above the threshold level throughout the duration of the cell culture.

In a further embodiment, the glucose concentration of the cell culture is monitored and the glucose containing feed or the glucose containing perfusion medium is added in order to maintain the glucose concentration in the culture medium above a threshold level. The threshold level should not lie below a glucose concentration that is critical for cell growth. The amount of glucose added should replenish the consumed glucose. However, high glucose concentrations in the cell culture leading to osmotic stress should be avoided.

In one embodiment, the glucose concentration in the cell culture is maintained above the threshold level of 5.5 mM (1 g/L). Upon drop of the glucose concentration below 5.5 mM (1 g/L) in the cell culture, the glucose containing feed or the glucose containing perfusion medium is added in an amount to result in a glucose concentration of up to 66 mM (12 g/L) in the cell culture.

In a preferred embodiment, the glucose concentration in the cell culture is maintained above the threshold level of 11 mM (2 g/L). Upon drop of the glucose concentration below 11 mM (2 g/L) in the cell culture, the glucose containing feed or the glucose containing perfusion medium is added in an amount to result in a glucose concentration of up to 44 mM (8 g/L) in the cell culture.

In a more preferred embodiment, the glucose concentration in the cell culture is maintained above the threshold level of 16.5 mM (3 g/L). Preferably, upon drop of the glucose concentration below 16.5 mM (3 g/L) in the cell culture, the glucose containing feed or the glucose containing perfusion medium is added in an amount to result in a glucose concentration of 33 mM (6 g/L) in the cell culture.

In another preferred embodiment, the glucose concentration is maintained above the threshold level for the duration of the cell culture.

In one embodiment, a feed comprising glucose and galactose or a perfusion medium comprising glucose and galactose is added to the cell culture. Galactose and Glucose may be present in the feed medium or the perfusion medium at a ratio of between 1:0.5 to 1:15, preferably at a ratio of between 1:1 to 1:10, more preferably at a ratio of between 1:1 to 1:4 (Galactose:Glucose). In one embodiment, Galactose and Glucose are present in the feed medium or the perfusion medium at a ratio of about 1:2.

In one embodiment, the glucose concentration is monitored and the feed comprising glucose and galactose or the perfusion medium comprising glucose and galactose is added depending on the glucose consumption of the cell culture. In another embodiment, the galactose concentration is monitored and the feed comprising glucose and galactose or the perfusion medium comprising glucose and galactose is added depending on the galactose consumption of the cell culture.

In one embodiment, the host cells expressing FVII are cultured for a time period of at least 7 days, preferably for a time period of at least 8 days, most preferably for a time period of at least 9 days. In another embodiment, the host cells expressing FVII are cultured for a time period of 7-14 days, preferably of 8-12 days, more preferably of 9-11 days.

The basal medium may be any standard commercially available cell culture basal medium comprising at least the minimal amounts of nutrients required for cell growth (for example CD-CHO AGT medium (Invitrogen)). In a preferred embodiment, the galactose concentration in the basal medium is at least 1 mM, preferably at least 2.5 mM, more preferably at least 10 mM. The galactose concentration in the basal medium may be between 2.5 mM and 60 mM, preferably between 10 mM and 60 mM, more preferably between 10 mM and 40 mM. In one embodiment, the basal medium is serum-free. In a preferred embodiment, the basal medium is chemically defined. In another preferred embodiment, the basal medium is free of proteins from animal or human origin. In a further preferred embodiment, the basal medium is protein-free.

The feed medium may be any standard commercially available feed medium comprising nutrients and supplements that are consumed during cell cultivation in high concentrations. Galactose and glucose may be provided in a separate carbon feed. In one embodiment, the feed medium is serum-free. In a preferred embodiment, the feed medium is chemically defined. In another preferred embodiment, the feed medium is free of proteins from animal or human origin. In a further preferred embodiment, the feed medium is protein-free.

The perfusion medium may be any standard perfusion medium. The perfusion medium may be identical to the basal medium. The galactose concentration in the perfusion medium may be between 2.5 mM and 60 mM, preferably between 10 mM and 60 mM, more preferably between 10 mM and 40 mM. In one embodiment, the perfusion medium is serum-free. In a preferred embodiment, the perfusion medium is chemically defined. In another preferred embodiment, the perfusion medium is free of proteins from animal or human origin. In a further preferred embodiment, the perfusion medium is protein-free.

After protein expression, the recombinant FVII may be purified from the cell culture. In one embodiment, the FVII is secreted into the medium and FVII may be purified from the supernatant. The purification process may therefore involve removal of the host cells and other solids from the cell culture. Such removal can, for example, be achieved by centrifugation or filtration. In one embodiment, the recombinant FVII is further purified by chromatography, such as size exclusion chromatography, affinity chromatography, hydrophobic interaction chromatography, ion exchange chromatography and/or multimodal chromatography. The purification process may further involve centrifugation, ethanol precipitation and/or dialysis. In one embodiment, FVII is concentrated after purification. The process may involve further separation and/or isolation steps.

The glycosylation pattern of FVII prepared according to the method of the present invention may be determined by any method disclosed in the art. Examples for such methods include the analysis of intact proteins or subunits by liquid chromatography and mass spectrometry (LC-MS), analysis using a top-down approach, a middle-down approach or a bottom-up LC-MS approach (e.g., Zhang et al. (2013), Chem Rev, 113(4): 2343-2394 and Catherman et al., (2014), 445(4): 683-693). The intact or subunit MS approach relates to a method of protein identification that uses MS for the determination of intact proteins (or proteins partially cleaved into large fragments/subunits) molecular mass, based on the obtained mass-to-charge (m/z) detected. Mixtures of proteins must first be separated by liquid chromatography or other separation methods prior to analysis by mass spectrometry. The top-down approach relates to a method of protein identification that uses the m/z selection of intact proteins followed by fragmentation and m/z separation in a second stage of mass spectrometry. Mixtures of proteins must first be separated by liquid chromatography or other separation methods prior to analysis by mass spectrometry. The middle-down approach relates to top-down proteomics applied to a protein that has been cleaved into a few large fragments/sub-units (Analyzing Biomolecular Interactions by Mass Spectrometry. Edited by Jeroen Kool, Wilfried M. A. Niessen. 2015. John Wiley & Sons). The bottom-up approach relates to a method of protein identification that uses proteolytic digestion before analysis by liquid chromatography and mass spectrometry. Proteins can be isolated by gel electrophoresis prior to digestion or, alternatively, the protein mixture is digested, usually by a specific enzyme, and the resulting peptides are separated and analyzed by liquid chromatography coupled in-line to MS for identification using tandem mass spectrometry (Definitions of terms relating to mass spectrometry (IUPAC Recommendations 2013). K. Murray, R. Boyd et. al. 2013. Pure Appl. Chem., Vol 85, No. 7, pp. 1515-1609).

In a preferred embodiment, the level of Xyl-Xyl-Glc-glycosylation at position Ser52 of FVII is determined by the middle-down approach. In a further embodiment relating to the middle-down approach, Xyl-Xyl-Glc-glycosylation at position Ser52 of FVII is determined, wherein the FVII considered for analysis is fucosylated at Ser60.

In one preferred embodiment, the recombinant FVII prepared by the method of the present invention exhibits less than 80% Xyl-Xyl-Glc-glycosylation at position Ser52. In a more preferred embodiment, the recombinant FVII prepared by the method of the present invention exhibits less than 70% Xyl-Xyl-Glc-glycosylation at position Ser52. In a further preferred embodiment, the recombinant FVII prepared by the method of the present invention exhibits less than 65% Xyl-Xyl-Glc-glycosylation at position Ser52. In another preferred embodiment, the recombinant FVII prepared by the method of the present invention exhibits less than 60% Xyl-Xyl-Glc-glycosylation at position Ser52.

Also provided herein is a composition of matter comprising host cell comprising an expression system expressing recombinant FVII and a cell culture medium as defined herein. In a preferred embodiment, the cell culture medium comprises galactose at a concentration effective to increase O-glycan heterogeneity at Ser52 of FVII and/or to reduce the occurrence of Xyl-Xyl-Glc-glycosylation at position Ser52 of FVII.

The present invention also relates to a recombinant FVII composition. In one embodiment, the occurrence of Xyl-Xyl-Glc-glycosylation at Ser52 of recombinant FVII in the composition is below 80%. In a preferred embodiment, the occurrence of Xyl-Xyl-Glc-glycosylation at Ser52 of recombinant FVII in the composition is below 70%. In a further preferred embodiment, the occurrence of Xyl-Xyl-Glc-glycosylation at Ser52 of recombinant FVII in the composition is below 65%. In another preferred embodiment, the occurrence of Xyl-Xyl-Glc-glycosylation at Ser52 of recombinant FVII in the composition is below 60%.

Further, a pharmaceutical composition comprising the FVII prepared according to the present invention is provided. In one embodiment, the pharmaceutical composition further comprises a pharmaceutically acceptable excipient. Non-limiting examples of pharmaceutically acceptable excipients are pH adjusting agents, buffering agents and tonicity adjusting agents.

Also, a pharmaceutical composition of the invention is formulated to be compatible with its intended route of administration. Examples of routes of administration include parenteral, e.g., intravenous, intradermal, subcutaneous, oral (e.g., inhalation), transdermal (topical); transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases; such as hydrochloric acid of sodium hydroxide. Further suitable excipients are known from the prior art. The preparation can be enclosed in ampoules, disposable syringes or multiple dose vials made of glass or plastic.

The FVII prepared according to the method of the present invention for use in the treatment of haemophilia A is also provided herein. Further medical indications that can be treated with FVII prepared according to the present invention include haemophilia B, blood loss associated with trauma and surgery, acquired haemophilia, congenital FVII deficiency and Glanzmann's thrombasthenia.

Recombinant FVII is advantageous for therapeutic application, as recombinant FVII has a reduced risk of pathogen contamination and as its production process is less elaborate in comparison to plasma derived FVII. However, recombinant FVII exhibits a different glycosylation pattern than plasma derived FVII. In contrast to plasma-derived FVII, recombinant FVII prepared according to methods disclosed in the prior art exhibits high levels of Xyl-Xyl-Glc-glycosylation at position Ser52 of recombinant FVII.

The method according to the present invention allows reducing the level of Xyl-Xyl-Glc-glycosylation at position Ser52 of recombinant FVII. Thereby, the O-glycan heterogeneity of FVII is increased at Ser52. The recombinant FVII prepared according to the method of the present invention exhibits a glycosylation pattern at Ser52 that is closer to the natural distribution of the three forms Xyl-Xyl-Glc, Xyl-Glc- and Glc- than recombinant FVII prepared by the methods disclosed in the prior art. Accordingly, recombinant FVII prepared by the method of the invention exhibits a glycosylation pattern that more closely resembles plasma-derived FVII.

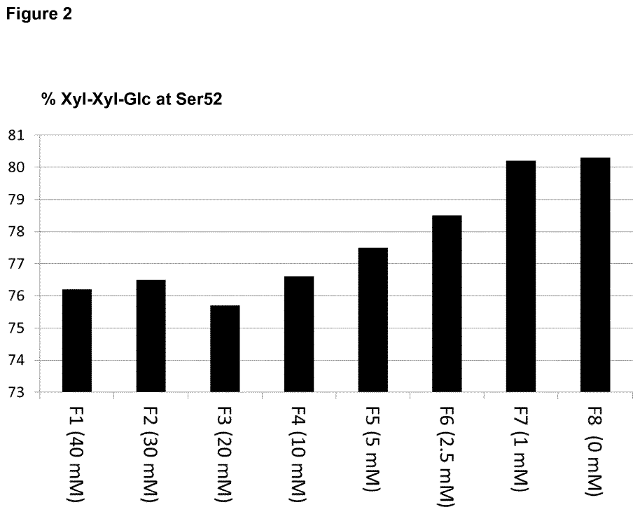

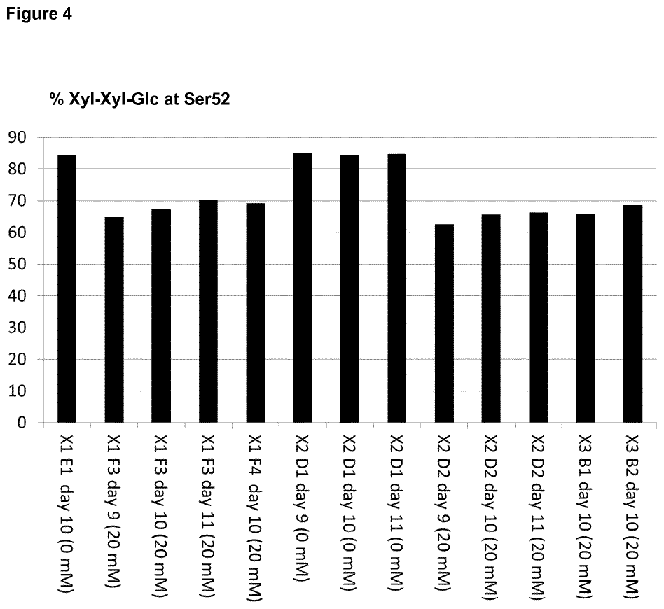

The present invention is based on the finding that the presence of galactose in the culture medium during expression of recombinant FVII results in an increase of O-glycan heterogeneity of FVII. Furthermore, the provided method results in a reduction of Xyl-Xyl-Glc-glycosylation at position Ser52 of recombinant FVII (FIG. 2 and FIG. 4).

The presence of galactose in the cell culture medium does not affect the viable cell density of the cultured cells. Cell viability is likewise not affected by the presence of galactose in the cell culture medium (Tables 2-4 and 7-9). The activity of recombinant FVII is not altered when FVII is expressed in the presence of galactose (Tables 5 and 11).

The FVII prepared according to methods of the present invention may exhibit reduced immunogenicity. In particular, patients receiving the FVII prepared according to method of the present invention are expected to less likely develop an unwarranted immune response. Furthermore, the patients receiving the FVII prepared according to method of the present invention are expected to less likely develop FVII inhibitors.

Hence, the present invention provides a method for preparing recombinant FVII exhibiting a glycosylation pattern that is more close to the glycosylation pattern of plasma derived FVII in comparison to FVII prepared by methods from the prior art. Furthermore, the FVII prepared by the method of the present invention exhibits increased protein stability.

### EXAMPLES

### Example 1: Determining Optimal Galactose Concentration for Increasing O-Glycan Heterogeneity of FVII

### Cell Culture

A Chinese Hamster Ovary (CHO) cell line that expresses recombinant human Factor VII fusion protein (rVII-FP) was created by transfecting CHO-S host cells (Invitrogen) using the GS expression system (Lonza). These cells were maintained in commercially available CD-CHO AGT medium (Invitrogen) supplemented with 50 μg/L reduced menadione sodium bisulfite (rMSB) (Richman), 25 μM methionine sulfoximine (MSX) (Sigma) and 1 mg/L insulin (Novo Nordisk). The seed train was maintained in shake flasks in a shaker incubator (Kuhner) maintained at 120 rpm, 37° C. with 8% CO₂ atmosphere and sub-cultured every 3 days to 3×10⁵ cells/mL.

Cells from the exponential growth phase of the cultures (at the end of regular 3-day passages) were used for the experiments. The appropriate amount of inoculum was determined for achieving 3×10⁵ cells/mL in 1 L non-baffled shake flasks (Corning) containing the seed train medium outlined above and supplemented with different levels of galactose (Sigma). The galactose concentration ranges tested were 1-40 mM with corresponding supplement-free negative control (for details of experimental setup and osmolality adjustment see Table 1). The shake flasks contained an initial culture working volume of 300 mL and are incubated in a shaker incubator operated at the same conditions as the seed train. The cultures were sampled on Day 0, 3 and 5 for cell count and product titer. The cultures were terminated on Day 5 and a bulk supernatant sample was also collected for O-glycosylation analysis.

TABLE 1

| | | | | | | Galactose Stock | | | Dilution & Osmolality Control Stock | | | | | |
|---|---|---|---|---|---|---|---|---|---|---|---|---|---|---|
| | | | | | | Theor- | | | Theor- | | | | | |
| | | | | | | etical | | | etical | | | | | |
| | | | | | | Osmol- | | Vol. | Osmo- | | | | | Vol. |
| | Culture | Glucose in | | | | ality | | to | lality | | | | | to |
| Flask | Volume | medium | | Galactose | | (mmol/ | | add | (mmol/ | Na | Na | K | K | add |
| # | L | mM | g/L | mM | g/L | kg) | g/L | (mL) | kg) | (mM) | (g/L) | (mM) | (g/L) | (mL) |
| 1 | 0.3 | 33.3 | 6.0 | 40 | 7.2 | 1111 | 200 | 10.80 | 1111 | 517 | 30.19 | 39 | 2.90 | 0.00 |
| 2 | 0.3 | 33.3 | 6.0 | 30 | 5.4 | 1111 | 200 | 8.10 | 1111 | 517 | 30.19 | 39 | 2.90 | 2.70 |
| 3 | 0.3 | 33.3 | 6.0 | 20 | 3.6 | 1111 | 200 | 5.40 | 1111 | 517 | 30.19 | 39 | 2.90 | 5.40 |
| 4 | 0.3 | 33.3 | 6.0 | 10 | 1.8 | 1111 | 200 | 2.70 | 1111 | 517 | 30.19 | 39 | 2.90 | 8.10 |
| 5 | 0.3 | 33.3 | 6.0 | 5 | 0.9 | 1111 | 200 | 1.35 | 1111 | 517 | 30.19 | 39 | 2.90 | 9.45 |
| 6 | 0.3 | 33.3 | 6.0 | 2.5 | 0.45 | 1111 | 200 | 0.68 | 1111 | 517 | 30.19 | 39 | 2.90 | 10.13 |
| 7 | 0.3 | 33.3 | 6.0 | 1 | 0.18 | 1111 | 200 | 0.27 | 1111 | 517 | 30.19 | 39 | 2.90 | 10.53 |
| 8 | 0.3 | 33.3 | 6.0 | 0 | 0 | 1111 | 200 | 0.00 | 1111 | 517 | 30.19 | 39 | 2.90 | 10.80 |

### Determination of Cell Growth and Viability

The cell density and viability in the culture were determined offline from a sample of culture using a Vi-CELL automated cell counter (Beckman Coulter).

TABLE 2
Total cell density (10⁶ cell/ml)

| | #1 | #2 | #3 | #4 | #5 | #6 | #7 | #8 |
|---|---|---|---|---|---|---|---|---|
| | (40 | (30 | (20 | (10 | (5 | (2.5 | (1 | (0 |
| Days | mM) | mM) | mM) | mM) | mM) | mM) | mM) | mM) |
| 0 | 0.27 | 0.33 | 0.22 | 0.32 | 0.28 | 0.30 | 0.25 | 0.23 |
| 3 | 2.25 | 2.66 | 2.17 | 2.21 | 2.09 | 2.29 | 2.39 | 2.34 |
| 5 | 5.31 | 4.84 | 5.15 | 4.88 | 4.54 | 4.25 | 4.31 | 4.40 |

TABLE 3
Viability (%)

| | #1 | #2 | #3 | #4 | #5 | #6 | #7 | #8 |
|---|---|---|---|---|---|---|---|---|
| | (40 | (30 | (20 | (10 | (5 | (2.5 | (1 | (0 |
| Days | mM) | mM) | mM) | mM) | mM) | mM) | mM) | mM) |
| 0 | 98.9 | 98.2 | 100.0 | 97.3 | 96.9 | 100.0 | 100.0 | 100.0 |
| 3 | 98.8 | 98.8 | 99.5 | 99.7 | 99.2 | 99.2 | 99.3 | 99.1 |
| 5 | 99.0 | 98.6 | 98.9 | 97.0 | 97.9 | 99.1 | 98.9 | 98.9 |

TABLE 4
Viable cell density (10⁶ cell/ml):

| | #1 | #2 | #3 | #4 | #5 | #6 | #7 | #8 |
|---|---|---|---|---|---|---|---|---|
| | (40 | (30 | (20 | (10 | (5 | (2.5 | (1 | (0 |
| Days | mM) | mM) | mM) | mM) | mM) | mM) | mM) | mM) |
| 0 | 0.27 | 0.33 | 0.22 | 0.31 | 0.27 | 0.30 | 0.25 | 0.23 |
| 3 | 2.23 | 2.63 | 2.16 | 2.20 | 2.08 | 2.27 | 2.37 | 2.32 |
| 5 | 5.26 | 4.77 | 5.09 | 4.73 | 4.44 | 4.21 | 4.27 | 4.35 |

The presence of galactose does not influence cell growth and viability.

### Quantification of Recombinant Factor VII Fusion Protein

Recombinant Factor VII fusion protein chromogenic activity in the cultures were determined from serially diluted supernatant samples using a commercially available chromogenic kit COASET Factor VII kit (Chromogenix). The assay was adapted for use with the Sysmex CS-5100 automated hemostasis analyzer (Siemens Healthcare) and performed according to manufacturers' instructions.

TABLE 5
Chromogenic activity (IU/L)

| | #1 | #2 | #3 | #4 | #5 | #6 | #7 | #8 |
|---|---|---|---|---|---|---|---|---|
| | (40 | (30 | (20 | (10 | (5 | (2.5 | (1 | (0 |
| Days | mM) | mM) | mM) | mM) | mM) | mM) | mM) | mM) |
| 0 | N/A | N/A | N/A | N/A | N/A | N/A | N/A | N/A |
| 3 | 6648 | 7167 | 6268 | 6406 | 6760 | 5449 | 5537 | 6220 |
| 5 | 16690 | 15992 | 12325 | 16192 | 16990 | 16567 | 15615 | 15162 |

The presence of galactose has no influence on the chromogenic activity of FVII.

### Analysis of FVII Glycosylation

### FVII Glycosylation Determined by Middle-Down Approach:

### Middle-Down LC-MS Scope

The middle-down liquid chromatography (LC) mass spectrometry (MS) assay is used as a characterisation assay for determining relative abundances of both O-linked glycoforms and γ-carboxylated glutamic acid (Gla) in the intact light chain from purified rVIIa-FP proteins that have been activated prior to this analysis. If unactivated, the use of tissue factor is optional in this procedure. The O-linked glycoforms at Ser52 and Ser60 are part of the Post Translational Modifications (PTMs) on the rVII-FP as are the γ-carboxylated (Gla) moieties, totaling a maximum of 10 in the Gla domain. The focus of this method described below is the determination of the relative abundance of the intact light chain subunit glycoforms at Ser52, considering a fucosylation at Ser60. The predominant fraction of FVII expressed in CHO cells is fucosylated at Ser60.

### Materials

Ammonium Bicarbonate (NH4HCO3, Urea, Dithiothreitol (DTT), Iodoacetamide (IAM), Formic Acid, Acetonitrile, PNGase F, Recombinant Tissue Factor Hemosil RecombiPlastin 2G (Instrumentation Laboratory company), Hemosil RecombiPlastin 2G Diluent (Instrumentation Laboratory company), ESI-L low concentration tune mix.

### Middle-Down LC-MS Method

PNGase F treatment, Reduction and Alkylation

The relative content of O-glycoforms of rVII-FP was determined by middle-down LC-MS analysis of the intact light chain subunit. N-linked glycans were removed from 50 μg of rVII-FP samples via incubation with 10 units of PNGase F for 2 hours at 37° C. (ensuring a maximum volume of protein solution used for digestion ≤300 μL). For bulk drug intermediate (BDI) samples, activation of rVII-FP was performed with 10 μL of reconstituted Recombinant Tissue Factor RecombiPlastin 2G (reconstituted using 400 μL of Hemosil RecombiPlastin 2G Diluent) and incubated for 15 minutes at room temperature. Samples were denatured using 4M Urea/0.2M Ammonium Bicarbonate buffer, and reduced using 9 mM DTT for 60 mins at 60° C. Alkylation of reduced samples was achieved using 45 mM IAM for 30 mins at room temperature in the dark with agitation. The alkylation was then quenched with 25 mM DTT for 30 mins at room temperature in the dark with agitation. Samples are then centrifuged at ˜16,000×g for 3 mins prior to transfer to a HPLC vial for analysis.

### Reversed-Phase Liquid Chromatography

Reversed-phase separation of N-deglycosylated, reduced and alkylated rVII-FP was carried out using an UltiMate 3000 HPLC system (Thermo Scientific) equipped with a Zorbax 300SB-C8 100×2.1 mm 3.5 μm column (Agilent technologies). Solvent A consisted of 0.1% FA in water, and solvent B included 90% ACN and 0.085% FA in water. The temperature was maintained at 25° C. and the flow rate set to 0.2 mL/min. After sample loading (amounts being ≥21 μmol) on column for 2 mins at 20% B, a linear gradient from 20% to 50% was run over 16 mins, followed by a column wash involving 50% to 80% in 2 mins. After washing for 2 min at 80% B, the gradient was set back to 20% B at 25 mins for re-equilibration for 5 mins. The protein signal was monitored by UV detection at 214 and 280 nm.

### Mass Spectrometry

A micrOTOF-Q II (Bruker Daltonics) Qq-TOF MS system was used in-line with the high-performance liquid chromatography (HPLC) system to identify the rVII-FP subunits. MS detection was performed in positive-ionization mode applying a capillary voltage of 4000 V with an end plate offset of 500 V. The dry gas flow rate was set at 8 L/min with a dry temperature of 180° C. and nebuliser gas rate of 1.5 bar. The ion transfer was optimized in the range m/z 50-3000 for highest sensitivity while keeping the resolution R>17,500 across the whole mass range. Spectra were acquired at a rate of 0.5 Hz. The instrument was calibrated in-line with each analysis using tune mix.

### Data Analysis

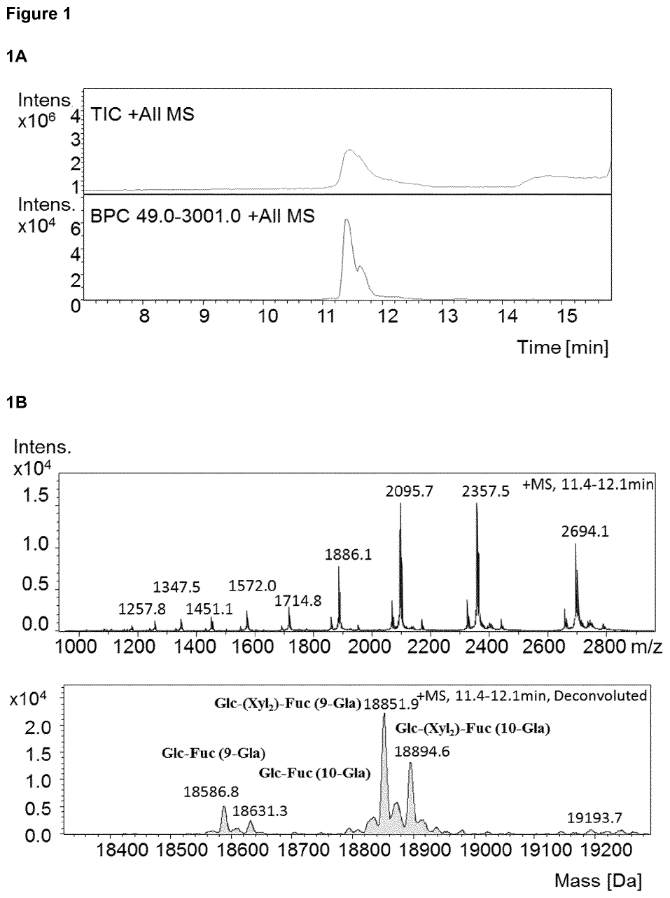

The typical retention time (RT) of the light chain in the Total ion Chromatogram (TIC) is between 11-13.0 mins. An averaged spectrum is generated across the determined chromatographic peak, which consists of predominately the 10- and 9-, γ-carboxylated (Gla) light chain forms of rVII-FP. This data is then transformed to the intact mass scale using DataAnalysis software (Bruker Daltonics) by MaxEnt deconvolution. FIG. 1A shows the TIC and the Base Peak Chromatogram (BPC) of the light chain; whilst FIG. 1B highlights the obtained average mass spectrum and corresponding deconvoluted and annotated spectrum, highlighting the major observed glycoforms. The nominal observed masses of ˜18,587 Da and ˜18,851 Da relate to the 9 Gla variant of Ser52-Glc and a Ser52-Glc-Xyl₂, respectively, all containing fucose at Ser60. The observed masses of ˜18,807 Da and ˜18,895 Da relate to the 8 Gla and 10 Gla variants with a Ser52-Glc-Xyl₂ and a fucose at Ser60. An additional O-linked glycan variant at Ser60 is observed at ˜19,507 kDa as a NANA-(Hex-HexNAc)-dHex-O-Ser60 (Ser60-tetra), which corresponds to the major Ser52-Glc-Xyl₂ structure. The obtained peak intensities of the deconvoluted light chain subunit mass signals listed by DataAnalysis (as shown in FIG. 1B) are utilised to determine relative ratios of Glc and Glc-Xyl₂ at Ser52 (considering fucosylation at Ser60) and expressed as a percentage. The calculation involves the distribution of O-linked glycans at Ser52 on the 9-Gla variant alone, or by determining the distribution of O-linked glycans at Ser52 on the major 9- and 10- Gla forms of rVII-FP LC.

TABLE 6
Results

| | | 9Gla only | | 10Gla only | | Combined using | |
|---|---|---|---|---|---|---|---|
| | | (n = 4) | | (n = 4) | | weighted mean (n = 4) | |
| | | Glc | Glc-Xyl₂ | Glc | Glc-Xyl₂ | Glc | Glc-Xyl₂ |
| | | (Ser52) | (Ser52) | (Ser52) | (Ser52) | (Ser52) | (Ser52) |
| Flask #1 | Average | 24.8 | 75.2 | 21.7 | 78.3 | 23.8 | 76.2 |
| (40 mM) | RSD | 1.5 | 0.5 | 3.5 | 1 | 1.8 | 0.6 |
| Flask #2 | Average | 24.7 | 75.3 | 21.1 | 78.9 | 23.5 | 76.5 |
| (30 mM) | RSD | 2.2 | 0.7 | 2.9 | 0.8 | 1.4 | 0.4 |
| Flask #3 | Average | 25.8 | 74.2 | 21.5 | 78.5 | 24.3 | 75.7 |
| (20 mM) | RSD | 1.5 | 0.5 | 2.4 | 0.6 | 1.2 | 0.4 |
| Flask #4 | Average | 25.1 | 74.9 | 20.5 | 79.5 | 23.4 | 76.6 |
| (10 mM) | RSD | 2.6 | 0.9 | 3 | 0.8 | 1.8 | 0.6 |
| Flask #5 | Average | 23.7 | 76.3 | 20.2 | 79.8 | 22.5 | 77.5 |
| (5 mM) | RSD | 2.7 | 0.8 | 1.9 | 0.5 | 1.2 | 0.4 |
| Flask #6 | Average | 23 | 77 | 18.7 | 81.3 | 21.5 | 78.5 |
| (2.5 mM) | RSD | 1.9 | 0.6 | 0.6 | 0.1 | 1.2 | 0.3 |
| Flask #7 | Average | 21.1 | 78.9 | 17.4 | 82.6 | 19.8 | 80.2 |
| (1 mM) | RSD | 3.7 | 1 | 2.6 | 0.5 | 2.8 | 0.7 |
| Flask #8 | Average | 21.3 | 78.7 | 17.2 | 82.8 | 19.7 | 80.3 |
| (0 mM) | RSD | 1.4 | 0.4 | 3.6 | 0.7 | 1.3 | 0.3 |

Results are also shown in FIG. 2.

### Conclusion

The results show that by increasing the galactose concentration in the medium, Xyl-Xyl-Glc-glycosylation of Ser52 of recombinant FVII is reduced (see Table 6 and FIG. 2).

### Example 2: Large Scale Expression of FVII in the Presence/Absence of Galactose

### Cell Culture

Chinese Hamster Ovary cell line that expresses recombinant human Factor VII fusion protein (rVII-FP) was created by transfecting CHO-S host cells (Invitrogen) using the GS expression system (Lonza). These cells were maintained in commercially available CD-CHO AGT medium (Invitrogen) supplemented with 50 μg/L reduced menadione sodium bisulfite (rMSB) (Richman), 25 μM methionine sulfoximine (MSX) (Sigma) and 1 mg/L insulin (Novo Nordisk). Cells were grown in shake flasks maintained at 37° C. with 8% CO₂ atmosphere and subcultured every 3 days to 3×10⁵ cells/mL.

Cells from the exponential growth phase of the cultures (at the end of regular 3-day passages) were used for the experiments. The appropriate amount of inoculum was determined for achieving 3×10⁵ cells/mL in the bioreactors containing 2.7 L of a medium based on the DMEM/F12 formulation. All cultures contain 1 mg/L insulin (Novo Nordisk) and starting glucose concentrations of 6 g/L, test cultures were further supplemented with starting galactose (Sigma) concentrations of 3.6 g/L.

Cultures used in the experiments were supplemented daily with rMSB (Richman) from Day 0 to Day 10, totalling 0.56 μM final concentration, to ensure availability to support proper gamma-carboxylation of the rVII-FP. In addition to the rMSB, the cultures were fed equal volumes (75 mL) of a nutrient feed solution daily from Day 3 to 10, totalling 20% of the initial culture volume, to prevent depletion of key amino acids and vitamins. A carbon feed consisting of 120 g/L galactose: 200 g/L glucose (according to ratio at start of culture) was also added based on glucose requirement to restore glucose concentration to 6 g/L whenever it falls below 3 g/L.

### Bioreactor Operations

The bioreactor cultures were conducted in 5 L double-walled, round-bottom glass vessels with a heated water jacket (Sartorius). The reactors were inoculated with a seeding density of 3×10⁵ cells/mL at a working volume of ˜3 L. Positive pressure was maintained in the bioreactor with a constant 150 mL/min air head-space sweep. The high dissolved oxygen concentration (DO) at inoculation was allowed to naturally drift down to the set-point level of 40% air saturation and then maintained at set-point via a 60 μm microsparger with constant sparge rate of 5 mL/min air and oxygen on demand. Agitation rate was set at 100 rpm using a 64 mm Rushton impeller. pH in the culture was maintained between 6.80 and 7.20 with broad dead-band control from inoculation to end of culture via intermittent carbon dioxide addition to the gas sparge or 2 M NaOH (Sigma-Aldrich) solution. Daily samples were collected aseptically from the bioreactor for determination of cell growth, viability, rVII-FP activity and some metabolites concentrations. Additional larger volume samples were also removed on Day 9, 10 and 11 of the cultures for further product characterization (eg. RP-HPLC protein quantification, O-glycosylation) after clean-up using a scale-down purification process.

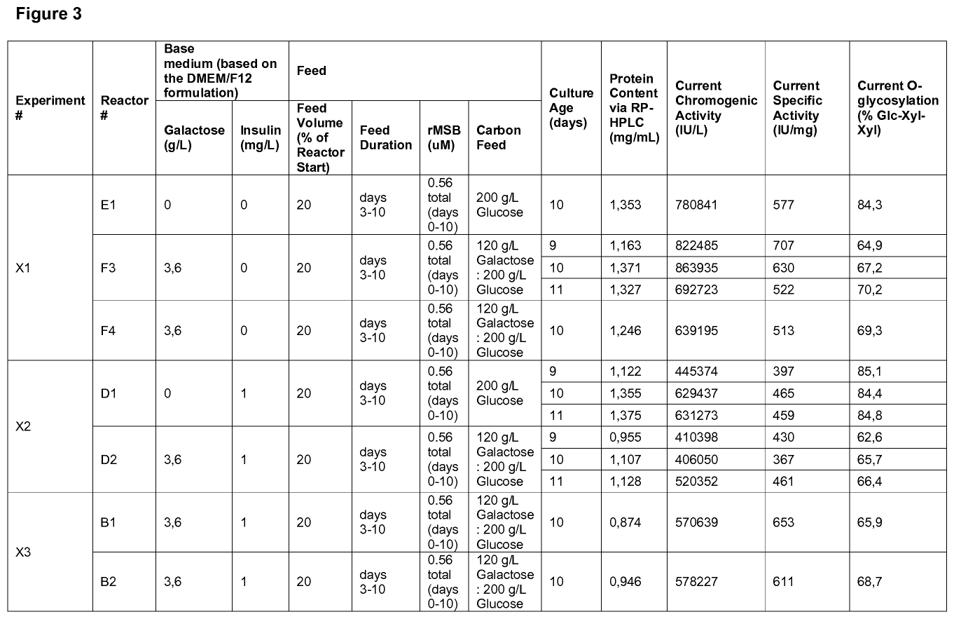

For overview of experimental outline (and results) see FIG. 3.

### Determination of Cell Growth and Viability

The cell density and viability in the culture were determined as described for Example 1.

TABLE 7
Total cell density (10⁶ cells/ml):

| | X1 E1 | X1 F3 | X1 F4 | X2 D1 | X2 D2 | X3 B1 | X3 B2 |
|---|---|---|---|---|---|---|---|
| Days | (0 mM) | (20 mM) | (20 mM) | (0 mM) | (20 mM) | (20 mM) | (20 mM) |
| 0 | 0.29 | 0.34 | 0.33 | 0.32 | 0.31 | 0.33 | 0.35 |
| 1 | 0.62 | 0.6 | 0.54 | 0.54 | 0.58 | 0.64 | 0.7 |
| 2 | 1.28 | 1.32 | 1.24 | 1.21 | 1.34 | 1.56 | 1.7 |
| 3 | 2.32 | 2.33 | 2.37 | 2.7 | 2.91 | 3.4 | 3.24 |
| 4 | 4.15 | 4.34 | 4.24 | 4.76 | 4.91 | 5.4 | 5.55 |
| 5 | 5.41 | 5.55 | 5.59 | 5.98 | 5.63 | 6.44 | 6.22 |
| 6 | 5.76 | 5.95 | 6.08 | 6.57 | 6.09 | 6.5 | 6.35 |
| 7 | 6.47 | 5.69 | 5.84 | 6.31 | 5.77 | 5.86 | 6.16 |
| 8 | 5.89 | 5.24 | 5.49 | 6.27 | 5.4 | 5.53 | 5.21 |
| 9 | 5.29 | 4.52 | 4.91 | 5.36 | 4.48 | 4.39 | 4.93 |
| 10 | 4.67 | 3.84 | 3.96 | 4.44 | 3.34 | 4.03 | 4.15 |
| 11 | 5.27 | 3.38 | 3.85 | 4.61 | 4 | 4.31 | 4.36 |
| 12 | 4.35 | 4.47 | 4.63 | 4.83 | 3.88 | 4.74 | 5.07 |

TABLE 8
Viability (%)

| | X1 E1 | X1 F3 | X1 F4 | X2 D1 | X2 D2 | X3 B1 | X3 B2 |
|---|---|---|---|---|---|---|---|
| Days | (0 mM) | (20 mM) | (20 mM) | (0 mM) | (20 mM) | (20 mM) | (20 mM) |
| 0 | 98.4 | 97.2 | 99 | 98.9 | 98.9 | 98.6 | 98.7 |
| 1 | 98.7 | 97.7 | 96.5 | 98.3 | 98.6 | 97.5 | 99.2 |
| 2 | 96 | 96.9 | 95.9 | 97.1 | 98.4 | 98.2 | 98.3 |
| 3 | 94.3 | 94.8 | 95.5 | 96.6 | 97.7 | 98.8 | 98.9 |
| 4 | 95.9 | 97.5 | 97.5 | 97.4 | 97.5 | 98 | 98.6 |
| 5 | 97.1 | 97.6 | 98.3 | 96.9 | 97.2 | 97.6 | 97.2 |
| 6 | 96.4 | 96.5 | 96.4 | 94.9 | 95.1 | 93.9 | 93.7 |
| 7 | 93.5 | 91.8 | 92.2 | 92.8 | 92.1 | 88.5 | 89.1 |
| 8 | 91.3 | 88.4 | 87.3 | 89.2 | 83.7 | 83.6 | 84.4 |
| 9 | 89.6 | 84.4 | 84.9 | 89.5 | 78.3 | 79.5 | 77.8 |
| 10 | 85.9 | 79.3 | 80.1 | 85 | 76.7 | 73.8 | 74.1 |
| 11 | 72.3 | 76.6 | 77.1 | 80.1 | 70.1 | 68.1 | 66.3 |
| 12 | 72.3 | 68.2 | 66 | 72.4 | 62 | 61.3 | 58.9 |

TABLE 9
Viable cell density (10⁶ cells/ml)

| | X1 E1 | X1 F3 | X1 F4 | X2 D1 | X2 D2 | X3 B1 | X3 B2 |
|---|---|---|---|---|---|---|---|
| Days | (0 mM) | (20 mM) | (20 mM) | (0 mM) | (20 mM) | (20 mM) | (20 mM) |
| 0 | 0.29 | 0.33 | 0.33 | 0.32 | 0.31 | 0.33 | 0.35 |
| 1 | 0.61 | 0.59 | 0.52 | 0.53 | 0.57 | 0.62 | 0.69 |
| 2 | 1.23 | 1.28 | 1.19 | 1.17 | 1.32 | 1.53 | 1.67 |
| 3 | 2.19 | 2.21 | 2.26 | 2.61 | 2.84 | 3.36 | 3.2 |
| 4 | 3.98 | 4.23 | 4.13 | 4.64 | 4.79 | 5.29 | 5.47 |
| 5 | 5.25 | 5.42 | 5.49 | 5.79 | 5.47 | 6.29 | 6.05 |
| 6 | 5.55 | 5.74 | 5.86 | 6.23 | 5.79 | 6.1 | 5.95 |
| 7 | 6.05 | 5.22 | 5.38 | 5.86 | 5.31 | 5.19 | 5.49 |
| 8 | 5.38 | 4.63 | 4.79 | 5.59 | 4.52 | 4.62 | 4.4 |
| 9 | 4.74 | 3.81 | 4.17 | 4.80 | 3.51 | 3.49 | 3.84 |
| 10 | 4.01 | 3.05 | 3.17 | 3.77 | 2.56 | 2.97 | 3.08 |
| 11 | 3.81 | 2.59 | 2.97 | 3.69 | 2.80 | 2.94 | 2.89 |
| 12 | 3.15 | 3.05 | 3.06 | 3.50 | 2.41 | 2.91 | 2.99 |

The presence of galactose does not influence cell growth and viability.

### Galactose Measurement

The galactose concentrations in the culture supernatant were determined offline from a sample of culture using a YSI 2700 biochemical analyzer (Nova Biomedical).

TABLE 10
Galactose (g/L)

| | X1 E1 | X1 F3 | X1 F4 | X2 D1 | X2 D2 | X3 B1 | X3 B2 |
|---|---|---|---|---|---|---|---|
| Days | (0 mM) | (20 mM) | (20 mM) | (0 mM) | (20 mM) | (20 mM) | (20 mM) |
| 0 | 0.000 | 3.55 | 3.26 | 0.004 | 3.5 | 2.73 | 2.77 |
| 1 | 0.000 | 3.36 | 3.37 | 0.000 | 3.44 | 2.77 | 2.78 |
| 2 | 0.000 | 3.47 | 3.29 | 0.000 | 3.36 | 2.91 | 2.9 |
| 3 | 0.000 | 3.31 | 3.12 | 0.000 | 3.22 | 2.76 | 2.65 |
| 4 | 0.000 | 2.98* | 2.86* | 0.000 | 2.79* | 2.5 | 2.55 |
| 5 | 0.000 | 4.12 | 3.94 | 0.000 | 4.49 | 2.28* | 2.3* |
| 6 | 0.000 | 3.86* | 3.6* | 0.003 | 4.18 | 4.32 | 4.65 |
| 7 | 0.010 | 5.44 | 5.31 | 0.007 | 3.86* | 4.04 | 4.42 |
| 8 | 0.018 | 4.82 | 4.92 | 0.000 | 5.68 | 3.6 * | 3.88* |
| 9 | 0.026 | 4.58* | 4.57* | 0.036 | 4.06 | 5.57 | 5.58 |
| 10 | 0.002 | 6.19 | 6.2 | 0.019 | 4.92* | 4.59 | 5.09 |
| 11 | 0.052 | 5.73 | 5.82 | 0.023 | 6.26 | 4.64* | 4.72* |
| 12 | 0.036 | 5.23 | 5.37 | 0.000 | 5.79 | 6.37 | 6.38 |

Carbon feed addition*

### Quantification of Recombinant Factor VII Fusion Protein

Recombinant Factor VII fusion protein chromogenic activity in the cultures was determined as described for Example 1.

Chromogenic activity of culture supernatants is shown in Table 11.

Chromogenic activity of purified samples is shown in FIG. 3.

TABLE 11
Chromogenic activity (IU/L)

| | X1 E1 | X1 F3 | X1 F4 | X2 D1 | X2 D2 | X3 B1 | X3 B2 |
|---|---|---|---|---|---|---|---|
| Days | (0 mM) | (20 mM) | (20 mM) | (0 mM) | (20 mM) | (20 mM) | (20 mM) |
| 0 | 774 | 692 | 687 | 696 | 579 | | |
| 1 | 1762 | 2108 | 2030 | 1696 | 1511 | 2182 | 3565 |
| 2 | 4066 | 5397 | 5047 | 4005 | 3749 | 5054 | 7062 |
| 3 | 8116 | 8357 | 8679 | 8294 | 7473 | 8679 | 10461 |
| 4 | 11946 | 14552 | 14556 | 13469 | 11088 | 14171 | 15129 |
| 5 | 2085 | 20258 | 21472 | 15206 | 12567 | 18744 | 17682 |
| 6 | 24639 | 25844 | 27447 | 24070 | 20539 | 26698 | 20847 |
| 7 | 31676 | 38240 | 34030 | 31341 | 22904 | 30258 | 31926 |
| 8 | 48981 | 49115 | 42255 | 43202 | 37674 | 39808 | 38357 |
| 9 | 66003 | 56473 | 67285 | 56817 | 48851 | 53010 | 53648 |
| 10 | 77628 | 63601 | 62099 | 68433 | 55048 | 55959 | 61665 |
| 11 | 69772 | 72394 | 61907 | 76929 | 59010 | 45650 | 52642 |
| 12 | 56253 | 67112 | 61324 | 61620 | 51445 | 38802 | 47864 |

The presence of galactose has no influence on the chromogenic activity of FVII.

### Analysis of FVII Glycosylation

FVII glycosylation was determined as described for Example 1. Results are shown in FIG. 3 and FIG. 4.

### Conclusion

The results show that the presence of galactose in the medium decreases Xyl-Xyl-Glc glycosylation of Ser52 of recombinant FVII (see FIG. 3 and FIG. 4).

1. A method for preparing a recombinant Factor VII (FVII) with altered glycosylation at Ser52, comprising:
a) providing host cells expressing recombinant FVII,
b) culturing the cells in a cell culture medium comprising galactose, and
c) obtaining the recombinant FVII from the cell culture,
wherein the recombinant FVII comprises increased O-glycan heterogeneity and/or reduced Xyl-Xyl-Glc-glycosylation at Ser52 as compared to recombinant FVII cultured in the absence of galactose, and wherein Ser52 corresponds to the serine at amino acid position 52 of SEQ ID NO:1.

2. The method of claim 1, wherein the method increases O-glycan heterogeneity at Ser52.

3. The method of claim 1, wherein the method reduces Xyl-Xyl-Glc-glycosylation at Ser52.

4. The method of claim 1, wherein galactose is present in the cell culture at a concentration effective to increase O-glycan heterogeneity at Ser52 of recombinant FVII and/or to reduce the occurrence of Xyl-Xyl-Glc-glycosylation at Ser52 of recombinant FVII.

5. The method of claim 1, wherein galactose is present in the cell culture at a concentration of at least 1 mM.

6. The method of claim 5, wherein galactose is present in the cell culture at a concentration of at least 10 mM.

7. The method of claim 6, wherein galactose is present in the cell culture at a concentration between 10 mM and 60 mM.

8. The method of claim 6, wherein galactose is maintained at a concentration of at least 10 mM in the cell culture for a time period of 7-14 days.

9. The method of claim 1, wherein the cell culture is a fed-batch culture and wherein galactose is present in a basal cell culture medium and/or in a feed medium.

10. The method of claim 9, wherein the galactose concentration in the basal medium is between 10 mM and 60 mM.

11. The method of claim 9, wherein the feed medium is added continuously or periodically during cell culture.

12. The method of claim 1, wherein the cell culture is a perfusion culture and wherein the culture medium comprising galactose is a basal cell culture medium and/or a perfusion medium.

13. The method of claim 1, wherein less than 80% of the recombinant FVII comprises Xyl-Xyl-Glc-glycosylation at Ser52.

14. The method of claim 13, wherein the occurrence of Xyl-Xyl-Glc-glycosylation at Ser52 of the recombinant FVII is determined by a middle-down approach.

15. The method of claim 14, wherein the recombinant FVII is fucosylated at Ser60.

16. The method of claim 1, wherein the cell culture medium is free of proteins from animal or human origin.

17. The method of claim 1, wherein the host cells are CHO cells.

18. (canceled)

19. A composition comprising recombinant Factor VII (FVII), wherein less than 80% of the recombinant FVII in the composition comprises Xyl-Xyl-Glc-glycosylation at Ser52 and wherein Ser52 of recombinant FVII is the residue corresponding to the serine at amino acid position 52 of SEQ ID NO:1.

20. The composition of claim 19, wherein the recombinant FVII is produced in CHO cells.

21. A cell culture composition comprising:
a) host cells expressing recombinant Factor FVII (FVII), and
b) cell culture medium comprising galactose at a concentration effective to increase O-glycan heterogeneity at Ser52 of the recombinant FVII and/or to reduce the occurrence of Xyl-Xyl-Glc-glycosylation at position Ser52 of recombinant FVII, wherein Ser52 of recombinant FVII is the residue corresponding to the serine at amino acid position 52 of SEQ ID NO:1.

22. A bioreactor comprising the cell culture composition of claim 21.

23. A recombinant FVII prepared by the method of claim 1.

24. A pharmaceutical composition comprising the recombinant FVII composition of claim 19 and a pharmaceutically acceptable excipient.

25. A method of treating or preventing hemophilia A or uncontrollable hemorrhage, comprising administering to a subject the recombinant FVII composition of claim 19.

26. The method of claim 1, wherein the recombinant FVII is a fusion protein.

27. The method of claim 1, wherein the recombinant FVII is an albumin fusion protein.

28. The method of claim 1, wherein galactose is present in the cell culture at a concentration of at least 2.5 mM.

29. The method of claim 6, wherein galactose is present in the cell culture at a concentration between 10 mM and 40 mM.

30. The method of claim 6, wherein galactose is maintained at a concentration of at least 10 mM in the cell culture for 8-12 days.

31. The method of claim 6, wherein galactose is maintained at a concentration of at least 10 mM in the cell culture for 9-11 days.

32. The method of claim 1, wherein less than 70% of the recombinant FVII comprises Xyl-Xyl-Glc-glycosylation at Ser52.

33. The composition of claim 19, wherein less than 70% of the recombinant FVII comprises Xyl-Xyl-Glc-glycosylation at Ser52.

34. The composition of claim 19, wherein the recombinant FVII is a fusion protein.

35. The composition of claim 19, wherein the recombinant FVII is an albumin fusion protein.


SEQUENCE LISTING (1 sequence)

SEQ ID NO: 1 - protein, 406 aa, Homo Sapiens [sequence omitted]